(19) United States
(12) Patent Application Publication
(10) Pub. No.: US 2025/0161592 A1
(43) Pub. Date: May 22, 2025

(54) VENTED PISTON

(71) Applicant: Haemograph Pty Ltd, Bayswater, Victoria (AU)

(72) Inventors: Alexander Samuel LUBANSKY, Bayswater, Victoria (AU); Robert A. NATURANI, Bayswater, Victoria (AU)

(73) Assignee: Haemograph Pty Ltd, Bayswater, Victoria (AU)

(21) Appl. No.: 18/836,659

(22) PCT Filed: Feb. 9, 2023
(86) PCT No.: PCT/AU2023/050082

§ 371 Date: Aug. 7, 2024

(30) Foreign Application Priority Data
Feb. 11, 2022 (AU) 2022900290

(51) Int. Cl. A61M 5/38; A61J 1/20; A61M 5/31; A61M 5/315

(52) U.S. Cl. CPC A61M 5/385; A61J 1/2072; A61M 5/31511; A61M 5/31513; A61M 2005/3123; A61M 2005/3128

(57) ABSTRACT

A vented piston is receivable within a fluid conduit. The piston includes a plunger body, the plunger body defining a fluid flow path extending between an upstream end and a downstream end; a fluid check-valve disposed across the fluid flow path, the fluid check-valve configurable to inhibit passage of gas from the downstream end to the upstream end of the fluid flow path and to inhibit passage of liquid from the upstream end to the downstream end of the fluid flow path; and a fluid flow inhibition member disposed across the fluid flow path to inhibit fluid flow within the fluid flow path, wherein the fluid check-valve is axially fixed relative to the plunger body.

### TECHNICAL FIELD

The present disclosure relates to vented pistons. More specifically, the present disclosure relates to vented pistons suitable for use in a syringe or fluidic system, for venting of gas while hindering outflow of liquid. The disclosure further relates to methods of filling and operating a syringe having a vented piston.

### BACKGROUND

Syringes are commonly used for handling of liquids, such as for testing or when administrating medications. Gas bubbles may be introduced to the syringe during filling of the syringe. For example, gas may be introduced during drawing up of medication from a vial. In many instances, the presence of gas bubbles in the syringe may be undesirable. Syringes may also be employed in fluidic systems such as microfluidic devices, in which removal of gas bubbles may be desirable.

Generally, to evacuate gas bubbles from a syringe, the user must invert the syringe and tap the syringe barrel to dislodge any bubbles from the base of the plunger. The user would then then depress the piston until all gas (and, frequently, a small volume of liquid) exits the needle. This process may be problematic in many scenarios, including but not limited to:
a. where capillary forces or fluid viscosity make removing bubbles from the syringe barrel difficult or impractical;
b. when syringe handling should be minimised, such as when dealing with dangerous materials; or
c. when use of an ordinary syringe might be difficult, such as when the operator suffers from an infirmity.

Further, this gas evacuation procedure may not be possible in the context of fluidic systems.

It is therefore desirable to provide a device for more easily evacuating bubbles from syringes or fluidic systems.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

According to one aspect of the present disclosure, there is provided a vented piston receivable within a fluid conduit, the piston comprising:
a plunger body, the plunger body defining a fluid flow path extending between an upstream end and a downstream end;
a fluid check-valve disposed across the fluid flow path, the fluid check-valve configurable to hinder passage of gas from the downstream end to the upstream end of the fluid flow path and to hinder passage of liquid from the upstream end to the downstream end of the fluid flow path; and
a fluid flow inhibition member disposed across the fluid flow path to inhibit fluid flow within the fluid flow path.

According to another aspect of the present disclosure, there is provided a vented piston receivable within a fluid conduit, the piston comprising:
a plunger body, the plunger body defining a fluid flow path extending between an upstream end and a downstream end, wherein the fluid flow path facilitates venting of fluid out of the plunger body;
a fluid check-valve disposed across the fluid flow path, the fluid check-valve configurable to inhibit passage of gas from the downstream end to the upstream end of the fluid flow path and to inhibit passage of liquid from the upstream end to the downstream end of the fluid flow path; and
a fluid flow inhibition member disposed across the fluid flow path to inhibit fluid flow within the fluid flow path.

The fluid check-valve may be axially fixed relative to the plunger body.

According to another aspect of the present disclosure, there is provided a vented piston receivable within a fluid conduit, the piston comprising:
a plunger body, the plunger body defining a fluid flow path extending between an upstream end and a downstream end;
a fluid check-valve disposed across the fluid flow path, the fluid check-valve configurable to inhibit passage of gas from the downstream end to the upstream end of the fluid flow path and to inhibit passage of liquid from the upstream end to the downstream end of the fluid flow path; and
a fluid flow inhibition member disposed across the fluid flow path to inhibit fluid flow within the fluid flow path,
   wherein the fluid check-valve is axially fixed relative to the plunger body.

The fluid flow path may facilitate venting of fluid out of the plunger body.

Any of the above aspects may further comprise one or more of the following features.

Venting of fluid out of the plunger body may comprise allowing fluid to exit the plunger body. The fluid flow path may facilitate venting out of the plunger body via the downstream end of the fluid flow path. This may be achieved, for example, by a plunger body comprising an open downstream end. The open downstream end may be in fluid communication with surrounding atmosphere and/or with a downstream portion of the fluid conduit.

The fluid flow inhibition member may be axially fixed relative to the plunger body and/or the fluid check valve. The fluid check valve may be axially fixed relative to the plunger body and/or the fluid flow inhibition member. In other embodiments, the fluid flow inhibition member may be axially movable relative to the plunger body and/or the fluid check valve. In some embodiments, the fluid flow inhibition member may be removable from the piston.

The fluid conduit may comprise an inner surface and the piston may form a fluidic seal with the inner surface of the fluid conduit. The piston may separate the fluid conduit into an upstream section and a downstream section. The fluid flow path may extend through the plunger body, fluidly connecting the upstream section and downstream section of the fluid conduit.

The plunger body may be adapted to form a fluidic seal with the inner surface of the fluid conduit. In some embodiments, the piston may further comprise a sealing member for forming a fluidic seal between the piston and an inner surface of the fluid conduit. For example, the sealing member may comprise a stopper at a distal end of the piston. The stopper may include an axial channel in fluid communication with the fluid flow path to fluidly connect the upstream and downstream sections of the fluid conduit. The stopper may be axially fixed relative to one or more of the plunger body, the fluid check valve and/or the fluid flow inhibition member.

In some embodiments, the plunger may have a side wall defining an internal lumen, which provides the fluid flow path. In some embodiments, the fluid flow path may generally extend along or parallel to an axis of elongation of the plunger body. In other embodiments, the fluid flow path may extend transverse to the axis of the plunger body or may be curvilinear or angled. The lumen may comprise one or more downstream openings defining the downstream end of the fluid flow path. The opening may comprise an open end of the lumen and/or an opening through the side-wall of the plunger, for example.

The fluid check valve may comprise a hydrophilic porous material and a hydrophobic porous material. Examples of such fluid check-valves have been disclosed in the present applicant's earlier filed patent application PCT specification PCT/AU2020/050902, the entirety of which is incorporated herein by reference.

The hydrophobic porous material may be disposed adjacent the hydrophilic porous material. One face of the hydrophilic porous material may be in fluid communication with the upstream end of the fluid flow path, and one face of the hydrophobic porous material is in fluid communication with the downstream end of the fluid flow path. The hydrophilic porous material may be configured to retain liquid from the upstream section to hinder passage of gas from the downstream section to the upstream section, and the hydrophobic porous material may be configured to inhibit passage of liquid from the upstream section to the downstream section.

The hydrophilic porous material may be disposed upstream of the hydrophobic porous material.

At least one of the hydrophilic porous material and the hydrophobic porous material may define a plurality of pores, and the plurality of pores have a median pore diameter in the range of about 0.003 microns to about 10 microns. In some embodiments, the plurality of pores have a median pore diameter less than about 1 micron, for example, about 0.005 microns. The hydrophilic porous material may define a plurality of first pores having a median second pore size less than about 0.5 microns. The hydrophobic porous material may define a plurality of second pores having a median second pore size less than about 0.3 microns.

In some embodiments, the hydrophobic porous material and the hydrophilic porous material may directly contact each other. In other embodiments, the hydrophobic porous material and the hydrophilic porous material are spaced from each other. For example, the hydrophobic porous material and the hydrophilic porous material may be spaced from each other by a distance greater than about 0 mm and less than about 2 mm. The hydrophobic porous material and the hydrophilic porous material may be separated by a material or medium that allows the passage or transmission of fluid. In some embodiments, the hydrophobic porous material and the hydrophilic porous material may be separated by a gap or a void.

The hydrophilic porous material may comprise a hydrophilic membrane. The hydrophobic porous material may comprise a hydrophobic membrane. In some embodiments, the hydrophobic membrane and/or the hydrophilic membrane comprises a polytetrafluoroethylene substrate. The fluid check-valve may be considered to comprise a dual layer hydrophobic and hydrophilic membrane system.

The hydrophilic porous material may comprise a hydrophilic coating. The hydrophobic porous material may comprise a hydrophobic coating. In some embodiments, the hydrophobic material may include a textured or patterned surface, wherein the texture or pattern of the surface inhibits wetting of the surface. In some embodiments, the hydrophilic material may include a textured or patterned surface, wherein the texture or pattern of the surface promotes wetting of the surface.

If the hydrophilic porous material has been exposed to a liquid, the fluid check-valve may inhibit the passage of gas from the downstream end to the upstream end with a backflow pressure limit of about −80 kPa, or about −90 kPA. In other embodiments, the backflow pressure limit may be significantly higher. A combination of membrane material and pore size may be selected to provide a desired backflow pressure limit.

The hydrophobic porous material may be configured to inhibit the passage of liquid from the upstream end to the downstream end with a leakage pressure limit of about 250 kPa, or about 400 kPa. In some embodiments, the hydrophobic porous material is configured to inhibit the passage of liquid from the upstream end to the downstream end with a leakage pressure limit of about 150 kPa. In some embodiments, the hydrophobic porous material is configured to inhibit the passage of liquid from the upstream end to the downstream end with a leakage pressure limit of about 100 kPa. The seal between the hydrophilic material and the plunger body may also be configured to withstand pressures at least up to the leakage pressure limit.

In some embodiments, the fluid check-valve may further comprise a retention body. The retention body may define a fluid aperture having an upstream side and a downstream side, wherein the hydrophilic and hydrophobic porous material are disposed to cover the fluid aperture.

The retention body may comprise a coupling member for connecting the retention body to the plunger body such that the fluid aperture is in fluid communication with the fluid flow path. For example, the coupling member may include a Luer connector.

The retention body may further comprise a seat for connecting to a sealing member for sealing between the piston and an inner surface of the fluid conduit.

The retention body may comprise a first part and a second part, connectable to each other to cooperatively retain the hydrophilic porous material and the hydrophobic porous material. The first part and second part may be: shaped to form a friction fit, or bonded together.

One of the first part and second part may be shaped to define a recess and a section of the other of the first and second parts is shaped to fit within the recess such that the first part and second part are coupled together. At least a section of each of the hydrophilic porous material and the hydrophobic porous material may be located within the recess. For example, in some embodiments, the first part may comprise a recess configured to receive the hydrophilic porous material and the hydrophobic porous material, and the second part may include a projection, receivable in the recess, and configured to retain the hydrophilic porous material and the hydrophobic porous material in the recess.

The fluid flow inhibition member may be positioned downstream of the fluid check-valve. In other embodiments, the fluid flow inhibition member may be positioned upstream of the fluid check-valve.

The fluid flow inhibition member may be configurable to selectively inhibit fluid flow within the fluid flow path. For example, the fluid flow inhibition member may be configurable between an open configuration in which fluid flow is substantially uninhibited and a closed (or sealing) configuration in which fluid flow is inhibited.

The fluid flow inhibition member may comprise a releasable, or breakable seal. In some embodiments, the fluid flow inhibition member may comprise a plug configured to seal the fluid flow path. That is, the plug may provide a fluid-tight seal within the fluid flow path, inhibiting passage of fluid past the plug. The plug may be at least partially receivable in the plunger body. For example, the plug may be receivable in a downstream end of the fluid flow path to seal the fluid flow path, that is, to inhibit fluid flow along the fluid flow path. The plug may be removable from the fluid flow path to allow fluid to flow along the fluid flow path. In other embodiments, the plug may comprise a pierceable, frangible or otherwise releasable seal.

In other embodiments, the fluid flow inhibition member may comprise a deformable portion at a downstream end of the plunger body. The deformable portion may include a flexible wall, for example. The flexible wall may be integral and/or formed in one piece with a side wall of the plunger body. The flexible wall may be movable between an open position and a closed position, in which the flexible wall at least partially obstructs the fluid flow path thereby to inhibit fluid flow. The flexible wall may be resiliently biased towards the open position. The flexible wall may be resiliently deformable into the closed position under application of compressive pressure (applied by a user's fingers, for example).

The fluid flow inhibition member may comprise a fluid flow control member. That is, the fluid flow inhibition member may be configurable to control the fluid flow within the fluid flow path. In some embodiments, the fluid flow inhibition member may be configurable to promote asymmetric flow within the fluid flow path. That is, the fluid flow inhibition member may be configured to inhibit fluid flow to a greater extent in one direction than in the opposite direction. For example, the fluid flow inhibition member may inhibit fluid back-flow in the upstream direction. In some embodiments, the fluid flow inhibition member may allow flow through the fluid flow path in one direction only. The fluid flow inhibition member may selectively seal or block the fluid flow path. The fluid flow inhibition member may be configurable to permit fluid flow from the upstream end to the downstream end of the fluid flow path and to inhibit fluid flow from the downstream end to the upstream end of the fluid flow path. Alternatively, or additionally, the fluid flow inhibition member may be configurable to permit fluid flow from the downstream end to the upstream end of the fluid flow path and to inhibit fluid flow from the upstream end to the downstream end of the fluid flow path.

The fluid flow inhibition member may comprise a valve. In some embodiments, the fluid flow inhibition member may comprise a passive valve. For example, the fluid flow inhibition member may comprise one or more of a diaphragm valve (e.g. a duckbill or slit valve), swing check valve, lift check valve, piston check valve, ball check valve, switchable check valve, spring valve or other suitable passive valve. In other embodiments, the fluid flow inhibition member may comprise an actuable valve, such as a tunable or switching valve (e.g. screw valve, butterfly valve, needle valve, gate valve). The valve may be configurable between an open configuration and a closed configuration. The valve may allow fluid flow in the fluid flow path while in the open configuration and may inhibit fluid flow in the fluid flow path while in the closed configuration.

In another embodiment, the fluid flow inhibition member may comprise an expandable balloon. The fluid flow inhibition member may further comprise a mesh which inhibits movement of the balloon in an upstream direction, but which allows the balloon to expand freely. In this embodiment, the balloon may trap gas which passes through the fluid check valve into the downstream end of the fluid flow path.

In still further embodiments, the fluid flow inhibition member may comprise a gas pump or a syphon in fluid communication with the fluid flow path.

In some embodiments, fluid may flow in the fluid flow path while the fluid flow inhibition member seals the fluid flow path (for example, when a plug is inserted in the fluid flow path or when a valve disposed across the fluid flow path is closed). In such embodiments, the fluid flow inhibition member may be positioned downstream of the fluid check-valve and may define a downstream cavity within the fluid flow path, between the fluid check-valve and the fluid flow inhibition member. The downstream cavity may have a volume configured such that gas may flow from the upstream end of the fluid flow path through the fluid check-valve and into the downstream cavity. The volume of the cavity may be selected to be larger than a total volume of gas to be evacuated from a syringe, for example. Gas (or other fluid) may be inhibited from flowing past the fluid flow inhibition member in either the upstream or downstream directions.

In some embodiments, the fluid flow inhibition member may comprise a closed downstream end of the fluid flow path. The fluid flow inhibition member may be integral with the plunger body. For example, the plunger body may comprise an opening at the upstream end of the fluid flow path and a blind end at the downstream end of the fluid flow path. Alternatively, the fluid flow inhibition member may comprise a plug or other seal, fixed across the fluid flow path.

In some embodiments, the fluid conduit may be comprised in a syringe. The syringe may include a syringe barrel defining the fluid conduit and adapted to partially receive the piston. When received in the syringe barrel, the piston may divide the syringe barrel into an upstream section and a downstream section. The syringe barrel may define a fluid opening adjacent the upstream section for inflow and outflow of fluid. In such embodiments, the piston may be movable within the syringe barrel to draw fluid into the syringe and evacuate fluid from the syringe. In other embodiments, the fluid conduit may be a pipe, a tube or other passage for fluid flow.

According to another aspect of the present disclosure, there is provided a vented syringe including a vented piston according to embodiments of the present disclosure.

According to another aspect of the present disclosure, there is provided a method of filling a vented syringe and purging gas from the vented syringe according to an embodiment of the present disclosure, the method comprising:
immersing the fluid opening in a liquid;
displacing the piston in a downstream direction to draw liquid into the upstream section of the vented syringe;
displacing the piston in an upstream direction, causing gas to flow through the fluid flow path in the downstream direction.

The gas may flow through the fluid flow path in the downstream direction and out of the syringe (i.e. purging the gas from the syringe via the downstream section). In other embodiments, the gas may be trapped in a downstream cavity within the piston and/or within the downstream section of the syringe.

The method may comprise further displacing the piston in the upstream direction to bring the fluid check-valve into contact with liquid. Once the check-valve has been wetted by the liquid, the check-valve is “activated” and inhibits passage of gas from the downstream end to the upstream end of the fluid flow path and inhibits passage of liquid from the upstream end to the downstream end of the fluid flow path.

In some embodiments, the vented syringe may be filled from a vial. In some embodiments, the method may include a step of pressurising the vial. For example, the method may further comprise, prior to displacing the piston in the downstream direction to draw liquid into the upstream section of the syringe barrel, displacing the piston in an upstream direction to force gas in the upstream section to flow out the fluid opening.

In some embodiments the method may further comprise, prior to immersing the fluid opening in the liquid source, displacing the piston in the downstream direction to cause gas to flow from the downstream section into the upstream section.

The method may further comprise displacing the piston in a downstream direction, after purging the gas, to draw further liquid into the upstream section of the syringe barrel.

The method may further comprise, prior to drawing liquid into the upstream section of the syringe barrel, drawing fluid (e.g. gas) into the upstream section of the syringe barrel.

In some embodiments, the method may further comprise, prior to displacing the piston in a downstream direction to draw liquid into the upstream section of the syringe barrel, configuring the fluid flow inhibition member to inhibit fluid flow (e.g. gas flow) from the downstream end to the upstream end of the fluid flow path (i.e. in the upstream direction).

In some embodiments, the method may further comprise, prior to displacing the piston in an upstream direction to cause gas to flow through the fluid flow path in the downstream direction, configuring the fluid flow inhibition member to allow fluid flow (e.g. gas flow) from the upstream end to the downstream end of the fluid flow path (i.e. in the downstream direction).

Configuring the fluid flow inhibition member to inhibit fluid flow may comprise configuring the fluid flow inhibition member to form a fluidic seal across the fluid flow path. Configuring the fluid flow inhibition member to allow fluid flow may comprise configuring the fluid flow inhibition member to break the fluidic seal across the fluid flow path. In some embodiments, where the fluid flow inhibition member comprises a plug, configuring the fluid flow inhibition member to inhibit flow in the upstream direction may comprise inserting the plug to seal the fluid flow path. In some such embodiments, configuring the fluid flow inhibition member to allow flow in the upstream direction may comprise removing, loosening or otherwise adjusting the plug to break the fluidic seal and open the fluid flow path. In other embodiments, configuring the fluid flow inhibition member to inhibit/allow fluid flow may comprise actuating a valve of the fluid flow inhibition member to open or close the valve, respectively. Actuating the valve may comprise manual and/or automatic actuation. Other actuation methods may include, but are not limited to, electronic, pneumatic and/or hydraulic actuation.

According to another aspect, there is provided a vented piston receivable within a fluid conduit, the piston comprising:
a plunger body, the plunger body defining a fluid flow path extending between an upstream end and a downstream end;
a fluid check-valve disposed across the fluid flow path, the fluid check-valve configurable to inhibit passage of gas from the downstream end to the upstream end of the fluid flow path and to inhibit passage of liquid from the upstream end to the downstream end of the fluid flow path; and
a fluid flow control member disposed across the fluid flow path and configurable to selectively inhibit fluid flow within the fluid flow path.

According to another aspect, there is provided a method of filling a vented syringe according to the present disclosure, the method including:
connecting the fluid opening with a liquid source;
causing the liquid to flow into the upstream section of the syringe barrel such that gas within the upstream section of the syringe barrel flows in a downstream direction through the fluid flow path.

The liquid may be caused to flow into the upstream section of the syringe barrel under pressure, for example, from an external pressure source (such as a pump). In some embodiments, the external pressure source provides automated filling pressure.

A position of the piston within the syringe barrel may be selected to define a fill volume. That is, the position of the piston may be selected to define a volume of the upstream section of the syringe barrel. The position of the piston relative to the barrel may be temporarily fixed during filling of the syringe.

The method may further comprise, after filling the vented syringe, displacing the piston in an upstream direction to dispense liquid from the vented syringe through the fluid opening.

In some embodiments, the fluid conduit may be comprised in a fluidic system, for example, a self-sealing venting fluidic system.

According to another aspect of the present disclosure, there is provided a fluidic system comprising:
a fluid conduit comprising an inner surface; and
a vented piston according to an embodiment of the present disclosure, wherein the piston is at least partially received within the fluid conduit and forms a fluidic seal with the inner surface of the fluid conduit to separate the fluid conduit into an upstream section to contain a gas and a liquid, and a downstream section to receive the gas.

The fluidic system may further comprise one or more control systems. For example, the fluidic system may comprise a motion control system, such as an automated motion control system, configured to engage the piston to control the movement of the piston. The automated motion control system may be configured to run a protocol to actuate the piston to move the piston in a downstream direction to draw fluid into the fluid conduit, actuate the piston to move the piston in an upstream direction to evacuate gas from the upstream section fluid conduit, actuate the piston to move the piston in a downstream direction to draw fluid further fluid into the upstream section to obtain a desired fluid volume, and actuate the piston to move the piston in an upstream direction to dispense a controlled volume of the fluid. Additionally or alternatively, the fluidic system may comprise a flow control system, such as an automated flow control system, for controlling the fluid flow inhibition member. The flow control system may configure the fluid flow inhibition member to inhibit fluid flow within the fluid flow path or to allow fluid flow within the fluid flow path. For example, the fluidic system may comprise a controller for actuating a gate or valve to open or close the gate or valve. The flow control system and the motion control system may form part of a single control system. For example, the fluidic system may comprise a control system configurable as a motion control system and/or as a flow control system. Alternatively, the motion control and flow control systems may be distinct control systems. In some embodiments, the fluidic system may form part of an automated fluid dispensing apparatus.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments are described in further detail below, by way of example, with reference to the accompanying drawings in which:

FIG. 1A is a schematic diagram of a vented piston according to one embodiment of the present disclosure;

FIG. 1B is an exploded schematic diagram of the vented piston of FIG. 1A;

FIG. 2A is a schematic diagram of a vented piston according to another embodiment of the present disclosure;

FIG. 2B is an exploded schematic diagram of the vented piston of FIG. 2B;

FIG. 3A shows a sequence of steps for drawing liquid from a vial using a prior art syringe;

FIG. 3B shows a sequence of steps for drawing up liquid using a vented syringe according to an embodiment of the present disclosure including the piston of FIG. 2A;

FIG. 4A shows a sequence of steps for automatic filling of a prior art syringe;

FIG. 4B shows a sequence of steps for automatic filling of a vented syringe according to an embodiment of the present disclosure, including the piston of FIG. 2A;

FIG. 5 is a graph showing results from a test of bubble purging at various angles of operation using a vented syringe including a piston according to an embodiment of the present disclosure; and

FIG. 6 shows a sequence of steps for drawing up liquid from a sealed vial, using a vented syringe according to an embodiment of the present disclosure including the piston of FIG. 2A.

### DESCRIPTION OF EMBODIMENTS

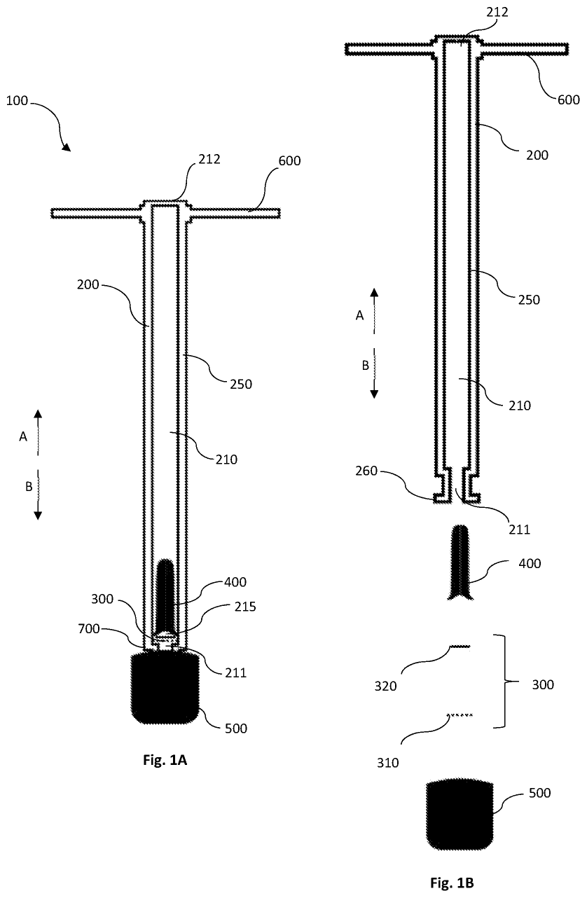

Referring to FIGS. 1A and 1B, an embodiment of a vented piston 100 according to the present disclosure is shown.

The vented piston 100 comprises a plunger body 200, a fluid check-valve 300 and a fluid flow inhibition member 400. The plunger body 200 defines a fluid flow path 210 extending between an upstream end 211 and a downstream end 212. The downstream end 212 may be open to atmosphere. As such, the fluid flow path 210 enables venting of gas from the piston 100. “Venting” in this context may be understood as meaning permitting escape of gas from within the fluid conduit and/or piston 100. The fluid check-valve 300 is disposed across the fluid flow path 210. The fluid check-valve 300 is configurable to inhibit passage of gas from the downstream end 212 to the upstream end 211 of the fluid flow path 210 and to inhibit passage of liquid from the upstream end 211 to the downstream end 212 of the fluid flow path 210. The fluid flow inhibition member 400 is also disposed across the fluid flow path 210 and is configured to selectively inhibit fluid flow within the fluid flow path 210.

In the embodiment of FIGS. 1A and 1B, the fluid flow inhibition member 400 is positioned downstream of the fluid check-valve 300, within the plunger body 200 in the fluid flow path 210. In other embodiments, the fluid flow inhibition member 400 may be positioned elsewhere in fluid communication with the fluid flow path 210.

In the illustrated embodiments, the fluid flow inhibition member 400 comprises a valve 400. The valve 400 promotes asymmetric flow within the fluid flow path 210. That is, the valve 400 permits fluid flow from the upstream end 211 to the downstream end 212 of the fluid flow path 210 and inhibits fluid flow from the downstream end 212 to the upstream end 211 of the fluid flow path 210. In some embodiments, the inhibition may prevent fluid flow from the downstream end 212 to the upstream end 211 of the fluid flow path 210. In other embodiments, the inhibition may partially prevent fluid flow from the downstream end 212 to the upstream end 211 of the fluid flow path 210, for example, such that gas flow in the upstream direction is subjected to greater resistance than flow in the downstream direction. Greater resistance to flow in the upstream direction may provide for more efficient evacuation of gas from the upstream section of the fluid conduit.

The valve 400 is disposed across the fluid flow path 210 such that fluid flowing through the fluid flow path must flow through the valve 400. As such, when the valve 400 is open, fluid may flow along the fluid flow path 210. Conversely, when the valve 400 is closed, fluid is inhibited from flowing along the fluid flow path 210. In the illustrated embodiments, the valve 400 is a passive valve. That is, the open or closed position of the valve 400 is responsive to the direction of fluid flow. The valve 400 is resiliently biased toward the closed position, inhibiting fluid flow, and remains closed against fluid back-pressure in the upstream direction. However, fluid pressure in the downstream direction opens the valve 400 such that fluid may flow from the upstream end to the downstream end.

The fluid flow inhibition member 400 may comprise one or more of a diaphragm valve (e.g. a duckbill or slit valve), swing check valve, lift check valve, piston check valve, ball check valve, switchable check valve, spring valve or other passive valve. In other embodiments, the fluid flow inhibition member may comprise an actuable valve, such as a tunable or switching valve (e.g. screw valve, butterfly valve, needle valve, gate valve).

In other embodiments, other suitable fluid flow control mechanisms may be used. For example, in some embodiments, the fluid flow inhibition member 400 may comprise a releasable seal. In some embodiments, the fluid flow inhibition member 400 may comprise a plug configured to seal the fluid flow path 210. The plug may be at least partially receivable in the plunger body 200. For example, the plug may be receivable in a downstream end of the fluid flow path 210 to seal the fluid flow path 210, that is, to inhibit fluid flow along the fluid flow path 210. The plug may be removable from the fluid flow path 210, loosenable, or otherwise adjustable to allow fluid to flow along the fluid flow path 210. The plug may comprise a rubber stopper, for example. In other embodiments, the fluid flow inhibition member 400 may comprise a seal which is pierceable, frangible or otherwise openable to allow fluid to flow along the fluid path 210, a solid or flexible cover, or a film. In some embodiments, the piston 100 may be configured to allow sealing of a downstream end 212 of the fluid flow path 210 by a user. For example, a fluid flow inhibition member 400 may be provided with the piston 100, or may be applicable to the piston 100 and/or removable from the piston 100 by a user. It is acknowledged that, in some cases, fluid flow inhibition may be achievable in the absence of a fluid flow inhibition member 400 by placement of a user's thumb across the fluid flow path 210. However, provision of a fluid flow inhibition member 400 to the piston 100 may advantageously improve usability and/or improve performance of the piston 100. For example, provision of a fluid flow inhibition member 400 to the piston 100 may reduce a number of steps performed by the user in operation of the piston 100. Further, provision of a fluid flow inhibition member 400 to the piston 100 may enhance consistency and/or predictability of performance of the piston 100, for example by reducing variability due to user technique. Provision of a fluid flow inhibition member 400 may allow for a longer storage time of medicament in a syringe comprising the piston 100. The fluid flow inhibition member 400 may provide a visible indicator of a state of the piston 100 and/or a syringe comprising the piston 100. For example, where the fluid flow inhibition member 400 comprises a plug, a user may be able to determine whether the syringe comprising the piston 100 has been used, based on whether the plug has been removed. In still further embodiments, the fluid flow inhibition member 400 may be applicable to the piston 100 and/or removable from the piston 100 by an electronic actuator (e.g. robotically).

In other embodiments, the fluid flow inhibition member 400 may comprise a deformable portion at a downstream end of the plunger body 200. The deformable portion may include a flexible wall, for example. The flexible wall may be integral and/or formed in one piece with a side wall of the plunger body 200. The flexible wall may be movable between an open position and a closed position, in which the flexible wall at least partially obstructs the fluid flow path 210 thereby to inhibit fluid flow. The flexible wall may be resiliently biased towards the open position. The flexible wall may be resiliently deformable into the closed position under application of compressive pressure (applied by a user's fingers, for example).

In another embodiment, the fluid flow inhibition member 400 may comprise an expandable balloon. The fluid flow inhibition member 400 may further comprise a mesh which inhibits movement of the balloon in an upstream direction, but which allows the balloon to expand freely. In this way, the balloon may trap gas which passes through the fluid check-valve 300 into the downstream end of the fluid flow path 210.

In still further embodiments, the fluid flow inhibition member 400 may comprise a gas pump or a syphon in fluid communication with the fluid flow path 210.

In other embodiments, fluid flow inhibition member 400 may be configured such that fluid flow within the fluid flow path 210 is partially inhibited. For example, the fluid flow inhibition member 400 may not seal the fluid flow path 210, but may provide increased resistance to fluid flow within the fluid flow path 210.

In other embodiments, the piston 100 may be configured such that fluid may still flow within the fluid flow path 210 while the fluid flow inhibition member 400 seals the fluid flow path 210. For example, fluid may be permitted to flow within the fluid flow path 210 when the valve 400 is in a closed configuration or, alternatively, when a plug or other sealing mechanism is sealing the fluid flow path 210. In some embodiments, the fluid flow inhibition member 400 may provide a substantially permanent fluidic seal across the fluid flow path 210. For example, the fluid flow inhibition member 400 may comprise a non-releasable seal or a plug fixed across the fluid flow path 210.

In such embodiments, the fluid flow inhibition member 400 may be positioned downstream of the fluid check-valve 300 and spaced from the fluid check-valve. A downstream cavity within the fluid flow path 210 may be defined between the fluid check-valve 300 and the fluid flow inhibition member 400. Where the downstream cavity has a sufficient volume, gas may flow in the downstream direction into the downstream cavity, even when the fluid flow path 210 is sealed by the fluid flow inhibition member 400.

In such embodiments, the fluid flow inhibition member 400 may partially prevent fluid flow within the fluid flow path 210, for example, such that fluid flow is subjected to greater resistance than if the fluid flow inhibition member 400 was not present. This partial inhibition of fluid flow may be configured to be sufficient to allow for generation of negative or positive pressure upon displacement of the plunger 100 within a fluid conduit, thereby allowing fluid to be displaced within the fluid conduit by the piston 100 (for example, allowing fluid to be drawn into or expelled from a syringe barrel).

The degree to which fluid flow within the fluid flow path is inhibited by the fluid flow inhibition member 400 may be influenced by a range of factors including, but not limited to, the volume of the downstream cavity.

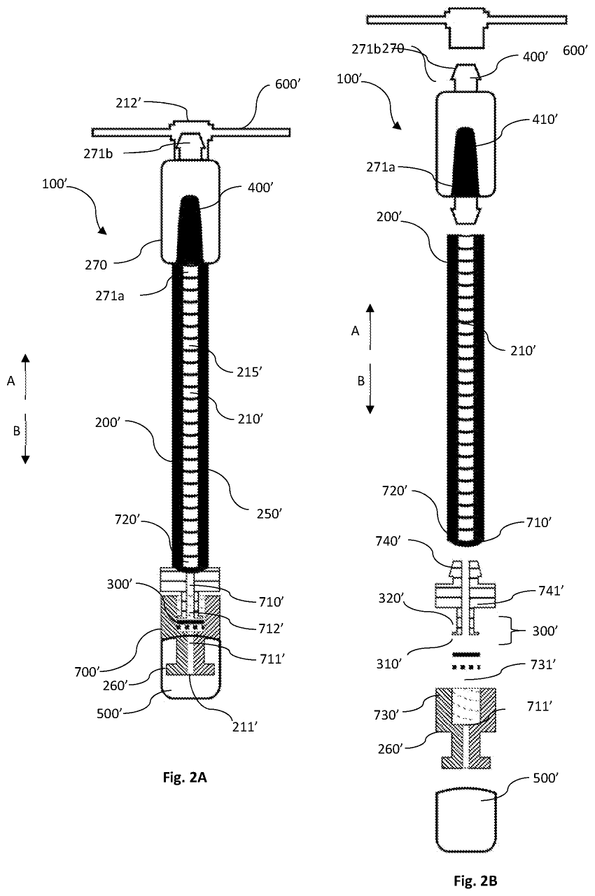

For example, in the embodiment of FIG. 1A, the valve 400 closely abuts the fluid check-valve 300. Nevertheless, a downstream cavity 215 is defined by the volume enclosed between the valve 400 and the fluid check-valve. In the embodiment illustrated in FIG. 2A, the valve 400′ is positioned further downstream of the check-valve 300, defining a downstream cavity 215′ within the lumen of the hollow plunger shaft 250′. The greater volume of the cavity 215′ compared to that of cavity 215 results in a lesser degree of inhibition of gas flow in the downstream direction. Thus in the embodiment of FIG. 2A, the valve 400 could be permanently closed (or replaced by a fixed plug or other sealing mechanism) and still allow gas to pass through the fluid check-valve 300 and into the downstream cavity 215′.

In some embodiments, the fluid flow inhibition member 400 may be axially fixed relative to one or more of the piston 100, stopper 500 and/or check valve 300. In other embodiments, the fluid flow inhibition member 400 may be axially movable relative to the piston 100, stopper 500 and/or check valve 300. For example, where the fluid flow inhibition member 400 comprises a seal or stopper, the fluid flow inhibition member 400 may be movable relative to the piston 100, stopper 500 and check valve 300 to insert and/or remove the seal or stopper from the piston.

In some embodiments, the fluid flow inhibition member 400 may be movable within the fluid flow path 210. For example, the fluid flow inhibition member 400 may be axially slidable along a portion of the fluid flow path 210, between an upstream position and a downstream position. In some embodiments, the movement of the fluid flow inhibition member 400 may be passive; that is, the fluid flow inhibition member 400 may move along the fluid flow path responsive to forces imparted from fluid flowing through the fluid flow path 210. In other embodiments, movement of the fluid flow inhibition member 400 may be actuated (or actuable), such that a user may control and/or adjust the movement of the fluid flow inhibition member 400.

In one example, a fluid flow inhibition member 400 may be movable in a portion of the fluid flow conduit 210, downstream of the fluid check-valve 300, between an upstream position adjacent the fluid check-valve 300 and a downstream position spaced further away from the fluid check-valve 300. A cavity may be defined between the fluid check-valve 300 and the fluid flow inhibition member 400, where the volume of the cavity is variable depending on the position of the fluid flow inhibition member 400 in the fluid flow path 210. As the piston 100 moves through the fluid conduit in an upstream direction, the fluid flow inhibition member 400 may be moved toward (or retained at) the upstream position adjacent to the fluid check-valve 300. This positioning of the fluid flow inhibition member 400 may minimise the volume of the cavity and the amount of fluid present in the cavity downstream of the fluid check-valve, and enhance creation of a vacuum. As the piston 100 is moved in the downstream direction, the fluid may impart forces on the fluid flow inhibition member 400 to move the fluid flow inhibition member 400 in the downstream direction towards the downstream position. This may increase the volume of the cavity downstream of the fluid check-valve.

Movement of the fluid flow inhibition member 400 within the fluid flow path 210 as described above may enhance, or provide additional flow asymmetry. However, such embodiments may be more complex and/or more expensive to manufacture.

The fluid check-valve 300 comprises a hydrophilic porous material 310 and a hydrophobic porous material 320 disposed adjacent the hydrophilic porous material 310. Examples of such fluid check-valves have been described in the present applicant's earlier filed PCT specification PCT/AU2020/050902. Fluid check-valves according to the present disclosure may incorporate one or more features of the fluid check valves described in PCT/AU2020/050902. In particular, the hydrophilic porous material 310 and/or hydrophobic porous material 320 may incorporate one or more features of the hydrophobic porous material and hydrophilic porous material described in PCT/AU2020/050902.

As shown in FIG. 1B, the hydrophilic porous material 310 is disposed upstream of the hydrophobic porous material 320. One face of the hydrophilic porous material 310 is in fluid communication with the upstream end 211 of the fluid flow path 210 and one face of the hydrophobic porous material 320 is in fluid communication with the downstream end 212 of the fluid flow path 210.

The fluid check-valve 300 initially allows passage of gas in both the upstream and downstream directions. However, when wetted, the fluid check-valve 300 inhibits passage of gas from the downstream end 212 to the upstream end 211 and inhibits passage of liquid from the upstream end 211 to the downstream end 212. That is, the hydrophilic porous material 310 is configured to retain liquid from the upstream end 211 to inhibit passage of gas from the downstream end 212 to the upstream end 211, and the hydrophobic porous material 320 is configured to inhibit passage of liquid from the upstream end 211 to the downstream end 212.

In the embodiment of FIGS. 1A and 1B, the fluid check-valve 300 is retained within the plunger body 200 at an upstream end of the fluid flow path 210. In other embodiments, the fluid check-valve 300 may be positioned elsewhere in fluid connection with the fluid flow path 210. The plunger body 200 comprises a retention body 700 for retaining the fluid check-valve 300. The plunger further comprises a hollow plunger shaft 250. The retention body 700 in this embodiment is integral with the plunger shaft 250. However, in other embodiments (such as that shown in FIGS. 2A and 2B), the retention body 700 and plunger shaft 250 may be separate components, wherein the retention body 700 is configured to be coupled to the plunger shaft 250 for conjoined movement therewith. The fluid flow path 210 extends through the retention body 700 and the plunger shaft 250.

The hydrophilic porous material 310 and the hydrophobic porous material 320 may be retained within the plunger body 200 by applying pressure, pinching, imprinting and deformation of the materials, fusing, ultrasonic welding, thermal welding, laser welding, overmolding, etc. Alternatively, the hydrophilic porous material 310 and the hydrophobic porous material 320 may be formed (e.g. injection molded, etched, patterned or laser formed) directly within the plunger body 200. The seal between the hydrophilic porous material 310 and the plunger body 200 must be able to withstand pressures at least up to the leakage pressure.

When the piston 100 is fit within a fluid conduit (such as a syringe barrel, for example), the piston 100 seals with the inner surface of the fluid conduit in a fluid-tight manner. That is, the seal formed between the piston 100 and the inner surface prevents fluid passing through the fluid conduit other than via the fluid flow path 210, through the fluid check-valve 300. As such, the fluidic seal between the piston 100 and the inner surface of the fluid conduit separates the fluid conduit into an upstream section and a downstream section.

Referring to FIG. 1A, the piston 100 may further comprise a sealing member such as stopper 500. In this embodiment, stopper 500 is positioned at an upstream end of the plunger body 200 and is configured to engage the inner surface of a fluid conduit to form a seal between the piston 100 and the fluid conduit. The stopper 500 forms a seal against the inner surface of the fluid conduit. In this embodiment, the plunger body 200 includes a connecting flange 260 for connecting to the stopper 500. However, in other embodiments, the stopper and plunger body may be connected by other suitable connection mechanisms. The stopper 500 includes an axial channel (not shown) providing fluid communication with the fluid flow path 210 and the upstream section of the fluid conduit.

In other embodiments, the piston 100 may include one or more O-rings or other similar sealing components. Such sealing components may be positioned at a distal end of the piston or elsewhere along the piston. Alternatively, one or more components of the piston (for example, the plunger, check valve or fluid flow inhibition member) may be configured (e.g. sized and/or shaped) to be close-fittingly receivable within the fluid conduit to engage an inner surface of the fluid conduit to seal the fluid conduit (create a fluidic seal), without requiring an additional sealing member.

The plunger 100 may be configured to be displaced along the fluid conduit while maintaining the seal with the fluid conduit. As such, the plunger may be actuated to drive liquid in the upstream section of the fluid conduit to move along the fluid conduit. The stopper 500 may be substantially rigidly connected to the plunger body 200, such that displacement of the piston 100 effects a corresponding and equal displacement of the stopper 500 within the fluid conduit. In the illustrated embodiments, the piston 100 includes a handle 600 to assist in the application of force to the piston 100 to displace the piston 100 within the fluid conduit.

In this embodiment, the piston 100, stopper 500, fluid flow inhibition member 400, and check valve 300 are configured for assembly together such that they are axially fixed and do not move relative to each other. That is, the piston 100, stopper 500, fluid flow inhibition member 400, and check valve 300 are configured for conjoined displacement within the fluid conduit. As the piston 100 is driven in an upstream direction B through the fluid conduit, the fluid flow inhibition member 400 allows liquid and gas to flow through the fluid flow path 210. The fluid check-valve 300 allows any gas within the upstream section of the fluid conduit to pass through the fluid check-valve 300 and flow through the fluid flow path 210. As the piston 100 is displaced in a downstream direction A through the fluid conduit, the fluid flow inhibition member 400 inhibits fluid flow through the fluid flow path 210. As such, displacement of the piston 100 in a downstream direction A creates a negative pressure, relative to atmospheric pressure, in the upstream section 921 of the fluid conduit due to the sealing of the stopper 500 against the fluid conduit and the closed position of the fluid flow inhibition member 400.

Once liquid contacts the fluid check valve 300, liquid may enter and be retained by the hydrophilic porous material 310. The hydrophilic porous material 310 with retained liquid inhibits and/or prevents the passage of gas through the hydrophilic porous material 310. The hydrophilic porous material 310 may, for example, have a strong capillary pressure to assist in retaining high surface tension liquids. As such, gas is prevented from flowing back to the upstream end of the fluid flow path 210. Liquid is inhibited from passing through the hydrophobic porous material 320. The hydrophobic porous material 320 may have a strong repelling pressure that inhibits high surface tension liquids from entering its structure. As such, as the piston 100 is driven in the downstream direction, gas is purged from the upstream section of the fluid conduit. Once the fluid check-valve is wetted by liquid from the upstream section, the fluid check-valve becomes “locked” and does not allow passage of liquid or gas through the fluid check-valve.

A vented piston 100′ according to another embodiment of the present disclosure is shown in FIGS. 2A and 2B. In this embodiment, the plunger body 200′ comprises a retention body 700′ and a hollow plunger shaft 250′. The fluid check-valve 300′ is held within retention body 700′. The retention body 700′ defines a fluid aperture 710′ having an upstream side 711′ and a downstream side 712′. The fluid flow path 210′ extends through the retention body 700′ and through the aperture 710′. The hydrophilic and hydrophobic porous materials 310′, 320′ are disposed to cover the fluid aperture 710′.

The retention body 700′ further comprises a coupling member 740′ for connecting the retention body 700′ to the distal end of the hollow plunger shaft 250′. In the illustrated embodiment, the hollow plunger shaft 250′ is hollow and the fluid flow path 210′ extends through a central lumen of the hollow plunger shaft 250′. The coupling member 720′ may be a Luer connector, or other such connector (for example, screw threads, press-fit, barbed tip, snap-fit or clip connection, flange, collar, revolute joint, ball joint, universal joint, cotterpin, knuckle joint or irreversible connections such as friction weld or glue), which allows for fluid communication along the fluid flow path 210′ between the retention body 700′ and the hollow plunger shaft 250′. In other embodiments, however, the plunger shaft may be solid. In such embodiments, the fluid flow path 210′ may terminate at a downstream portion of the retention body 700′, for example.

As shown in FIG. 2B, the retention body comprises a first part 730′ and a second part 740′, connectable to each other to cooperatively retain the hydrophilic porous material 310′ and the hydrophobic porous material 320′ therebetween. The first part 730′ comprises a recess 731′ configured to receive the hydrophilic porous material 310′ and the hydrophobic porous material 320′, and the second part 740′ includes a projection 741′ receivable in the recess 731′ and configured to retain the hydrophilic porous material 310′ and the hydrophobic porous material 320′ in the recess. The reverse configuration (that is, a recess in the second part 740′ and projection in the first part 730′) is also contemplated. In the illustrated embodiments, the retention body 700′ further comprises a connecting flange 260′ for connecting to the stopper 500′. However, the stopper may alternatively be connected to the retention body 700′ by other suitable connecting means.

In some embodiments, a friction fit is formed between the first part 730′ and the second part 740′. The shape of the recess 731′ and the first and second parts 730′, 740′ may assist in forming a friction fit. Additionally or alternatively, the first part 730′ or the second part 740′ may be coupled together by any one of: chemical bonding, heat sealing and an adhesive. The first part 730′ or the second part 740′ may be either removably or fixably attached to each other.

In some embodiments, the hydrophilic porous material 310′ and the hydrophobic porous material 320′ may be retained by the retaining body by applying pressure, pinching, imprinting and deformation of the materials, fusing, ultrasonic welding, thermal welding, laser welding, overmolding, etc. Alternatively, the hydrophilic porous material 310′ and the hydrophobic porous material 320′ may be formed (e.g. injection molded, etched, patterned or laser formed) directly within one or more of first part 730′ or the second part 740′.

In the embodiment of FIGS. 2A and 2B, the plunger body 200′ further comprises a housing 270 in which the fluid flow inhibition member valve 400′ is located. The housing 270 includes a pair of connectors 27la, 271b for connecting to the hollow plunger shaft 250′ and to the handle 600′. The connectors 271a, 271b may be Luer connectors, or similar, allowing for fluid flow therethrough such that the fluid flow path 210 is extended through the housing 270′ and the handle 600′.

The embodiment of FIGS. 1A and 1B appreciably requires fewer component parts than the embodiment of FIGS. 2A and 2B and may therefore be simpler and/or less expensive to manufacture.

### Syringes

In some embodiments, the vented piston 100, 100′ may be comprised in a vented syringe 900. Vented syringes according to embodiments of the present disclosure may be useful in controlled dosing applications (controlled dosing applications being those where the properties or quantity of a fluid are be defined by a syringe device). Vented syringes according to embodiments of the present disclosure may be used in various applications, including but not limited to medicament syringes and microfluidic devices.

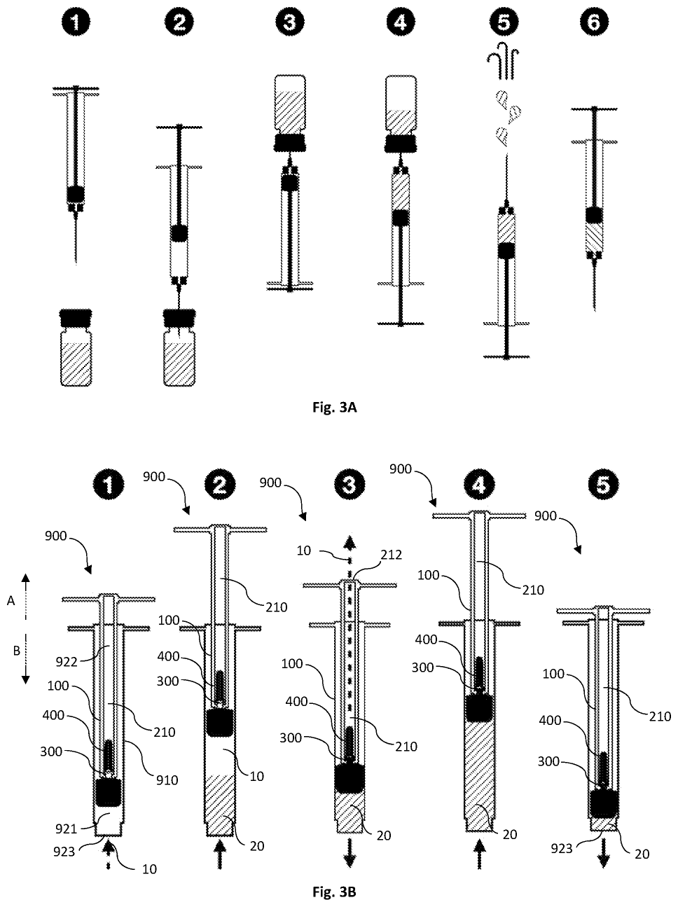

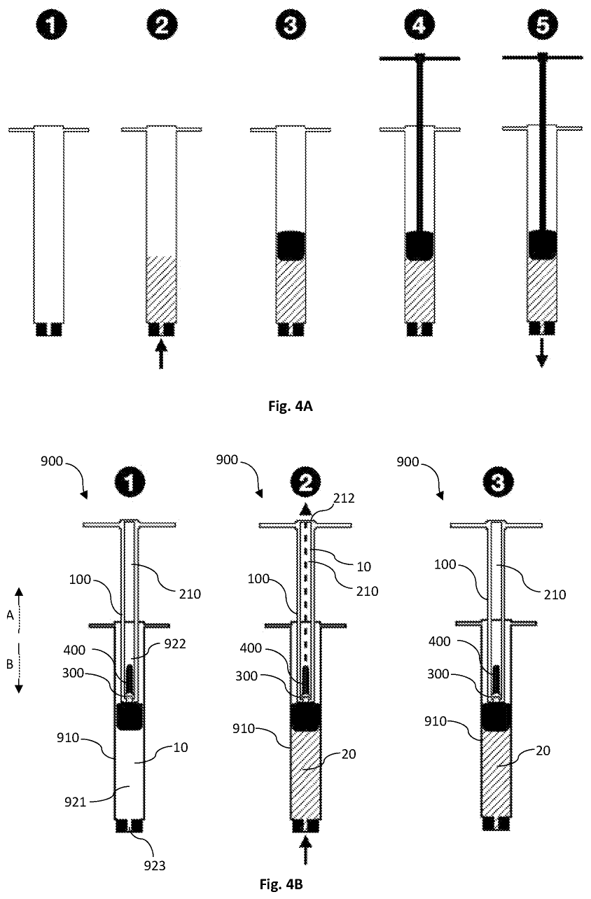

One example of such a vented syringe 900 is shown in FIGS. 3B and 4B. The vented syringe 900 incorporates the vented piston 100, but could alternatively include the vented piston 100′, or other embodiments of a vented piston described herein.

The vented syringe 900 comprises a barrel 910 defining the fluid conduit 920 having an upstream section 921 and a downstream section 922. A fluid opening is provided in the upstream section 921 to allow fluid to enter and exit the vented syringe. The piston 100 is partially received in the vented syringe barrel 910 such that the stopper 500 forms a fluidic seal with an inner surface of the barrel 910. The handle 600 facilitates displacement of the piston along the vented syringe barrel 910. The piston 100 may be displaced along the barrel 910 while maintaining the seal between the stopper 500 and the inner surface of the barrel 910.

In this embodiment, the piston 100, stopper 500, fluid flow inhibition member 400, and check valve 300 are configured to be axially fixed to one another for conjoined displacement within the barrel 910 of the syringe 900. Due to the seal between the piston 100 (via stopper 500) and the inner surface of the barrel 910, and the conjoined movement of the piston 100, stopper 500, fluid check valve 300 and valve 400, displacement of the piston 100 may result in fluid being drawn into or expelled from the fluid opening 923. That is, when the piston 100 is displaced in a downstream direction A through the syringe barrel 910, the fluid flow inhibition member 400 inhibits fluid flow through the fluid flow path 210. As such, displacement of the piston 100 in a downstream direction A creates a negative pressure in the upstream section 921 of the syringe barrel 910, relative to atmospheric pressure, due to the sealing of the stopper 500 against the inner surface of the barrel 910. This negative pressure may be used to draw liquid into the upstream section 921 of the barrel 910. When the check-valve 300 is not locked and the piston 100 is driven in an upstream direction B through the barrel 910, the valve 400 allows gas to flow through the fluid flow path 210. The fluid check-valve 300 allows any gas within the upstream section of the fluid conduit 920 to pass through the fluid check-valve 300 and flow through the fluid flow path 210. Liquid is allowed to flow through the fluid flow path upstream of the position of the check-valve 300 but is inhibited from flowing past the position of the check-valve 300. Once the liquid contacts the check-valve 300 and the check-valve 300 is wetted and locked, the piston 100 may be further displaced in the upstream direction B to expel fluid from the fluid opening 923.

Steps for drawing up medication from a vial and injecting the medication using a conventional syringe are illustrated in FIG. 3A. In FIG. 3A-1, a volume of air is drawn into the syringe. In FIG. 3A-2, the vial is penetrated with the syringe tip and the vial is pressurised with air. The vial and syringe are then inverted, as shown in FIG. 3A-3. Fluid from the vial is then drawn into the syringe barrel by withdrawing the plunger as shown in FIG. 3A-4. The plunger is depressed to eject air bubbles and correct the dosing volume, as shown in FIG. 3A-5. FIG. 3A-6 shows the syringe ready to proceed with an injection.

The four main pathways of drug injection are: intravenous, intrathecal, intramuscular, and subcutaneous. Injection of gas bubbles may be dangerous in some circumstances. Air injected directly into a vein may travel to the heart, lungs or brain where it may cause a heart attack, respiratory failure or stroke. Air injected into the spinal cord may cause intense headaches. By contrast, air bubbles injected into intra-cuticle and intramuscular tissue may be absorbed harmlessly (unless the user accidentally pierces a vein).

Needle localisation may be checked by slightly aspirating fluid from the injection site. If the aspirated fluid is red, the needle has penetrated a vein. If the aspirated fluid is clear or yellowish then the needle tip is subcutaneous or intramuscular. This practice is especially important when working with potent medications, for example local anaesthetics, which can cause heart complications if imperfectly injected in some patients.

During medication administration it is good practice and often essential to purge air from a syringe barrel after medication has been aspirated, as shown in FIG. 3A-5. During purging a small amount of medication is ejected from the syringe (if the user has correctly aspirated the required dose). However, some users may not be so skilful and will aspirate significantly more to ensure they can purge the syringe while also having the required dose.

Incorporating a vent in a syringe may avoid the need for a purging step which wastes medication. However, including a gas vent in the syringe design can create risks when a syringe is subject to ordinary use. For example, if gas passes back into the syringe during use, there is a risk of injecting air into the patient. Further, if the syringe needs to be aspirated prior to the injection step, there is a possibility that gas will enter the syringe barrel. Vented syringes according to embodiments of the present disclosure include a fluid check-valve, which inhibits gas from re-entering the upstream section of the syringe barrel once the fluid check-valve is wetted.

Further, simple syringe loading is an important part of making a syringe easy to use. For routine and generic applications, a traditional syringe will be loaded by the user by submerging the head of the syringe into a liquid, then manually withdrawing the piston/plunger until the liquid has adequately filled the barrel. Vented syringes according to embodiments of the present disclosure incorporate a fluid flow inhibition member for selectively inhibiting flow within the fluid flow path to create flow asymmetry. That is, the fluid flow inhibition member 400 may inhibit flow in one direction while allowing flow in the other direction along the fluid flow path. This allows the syringe to be easily loaded by simple withdrawal of the piston.

FIG. 3B illustrates a method for filling a vented syringe 900 according to an embodiment of the present disclosure. The method illustrated in FIG. 3B may be appropriate for filling the syringe from a reservoir of fluid, such as an unsealed vial for example.

In FIG. 3B-1, gas (e.g. air) 10 is drawn into the vented syringe barrel 910 by displacing the piston 100 in the downstream direction A. During this displacement, the fluid flow inhibition member 400 remains substantially closed. As such, displacement of the piston 100 creates a negative pressure in the upstream section 921 of the vented syringe barrel 910, drawing fluid into the upstream section 921 through the fluid opening 923.

In FIG. 3B-2, the vented syringe 900 is partially inserted into a source of liquid 20 (such as a vial of medication, for example) such that the fluid opening 923 is immersed within the liquid 20. The piston 100 is further displaced in the downstream direction A, drawing liquid 20 into the upstream section 921 of the vented syringe 900, as indicated by the arrow in FIG. 3B-2. In some cases, jetting of the liquid 20 may occur as the liquid 20 flows in to the upstream section 921 of the vented syringe 900. If a jet of liquid 20 contacts the fluid check-valve 300, the fluid check-valve 300 may become locked. To ameliorate the risk of premature locking of the fluid check-valve 300, the piston 100 may be moved away from the fluid opening 923 in step 3B-1 by a distance adequate to prevent the jet of liquid 20 contacting the fluid check-valve, prior to drawing liquid 20 into the upstream section 921.

As shown in FIG. 3B-3, the piston 100 is then depressed in the upstream direction B. As the piston 100 travels along the vented syringe barrel 910, the positive pressure opens the valve 400, allowing gas 10 to flow along the fluid flow path 210 through the fluid check-valve 300. The gas 10 is vented out of the syringe 900 via the open downstream end 212 of the hollow plunger rod of the piston 100. This step may substantially eliminate any bubbles of gas 10 from the upstream section 921.

Once the piston reaches the liquid 20, liquid 20 flows through the fluid flow path 210 until it contacts the fluid check-valve 300, wetting the hydrophilic membrane 310. Once the fluid check-valve membranes 310, 320 are wetted, the fluid check-valve may be considered “locked”. That is, the fluid check-valve will inhibit or prevent liquid 20 from flowing past the fluid check-valve 300 in the downstream direction, and will inhibit or prevent gas 10 from flowing past the fluid-check valve in the upstream direction B. Further displacement of the piston 100 in the upstream direction B therefore drives the liquid 20 out of the fluid opening 923.

Further liquid 20 may then be aspirated into the vented syringe 900 by displacing the piston 100 in the downstream direction A until a desired quantity of liquid 20 has entered the upstream section 921, as shown in FIG. 3B-4. Liquid 20 may be dispensed from the vented syringe when required by depressing the piston 100 to force the liquid 20 from the fluid opening, as shown in FIG. 3B-5.

In some embodiments, the vented syringe 900 may be filled from a sealed vial. In some embodiments, the method may include a step of pressurising the vial. In such embodiments, the method may comprise a step, prior to displacing the piston 100 in the downstream direction A to draw liquid 20 into the upstream section 921 of the syringe barrel 910, of displacing the piston 100 in an upstream direction B to force gas 10 in the upstream section 921 to flow out the fluid opening 923 and into the vial.

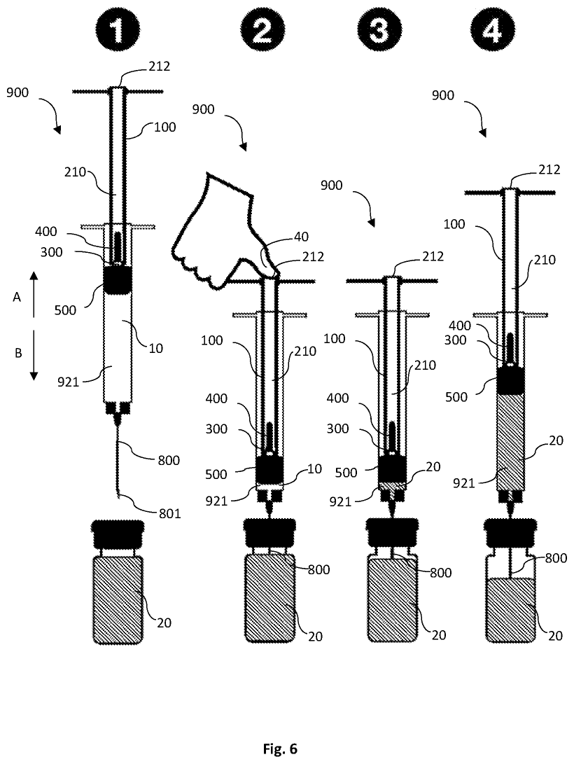

One example method of filling the vented syringe 900 from a sealed vial is illustrated in FIG. 6. In FIG. 6-1, gas (e.g. air) 10 is drawn into the vented syringe barrel 910 by displacing the piston 100 in the downstream direction A. During this displacement, the fluid flow inhibition member 400 remains substantially closed. As such, displacement of the piston 100 creates a negative pressure in the upstream section 921 of the vented syringe barrel 910, drawing gas into the upstream section 921 through the fluid opening 923. The volume of gas drawn into the upstream section 921 in this step may be configured with respect to a desired volume of liquid 20 to be extracted from the vial. For example, the volume of gas drawn into the upstream section 921 may be at least equal to, or greater than the desired volume of liquid 20. Alternatively, the volume of gas drawn into the upstream section 921 may be less than the desired volume of liquid 20.

As shown in FIG. 6, the syringe 900 may include (or be configured for attachment to) a needle cannula 800. The needle cannula 800 may be attached or attachable to the upstream end of the syringe 900. The needle cannula 800 may extend the fluid flow path 920 to an upstream end 801 of the needle cannula 800.

FIG. 6-2 shows a step of pressurising the vial. In this step, the needle cannula 800 is partially inserted into the sealed vial, for example by penetrating a seal of the vial such that the upstream end 801 of the needle cannula is positioned within the vial and immersed within the liquid 20 (such as liquid medicament, for example) contained in the vial. The piston 100 is then displaced in an upstream direction B to force gas 10 in the upstream section 921 to flow out the fluid opening 923 and into the vial. It will be appreciated that larger volumes of gas drawn into the upstream section 921 of the syringe 900 in step 6-1 may result in correspondingly higher pressure in the vial after the pressurisation step 6-2.

In some embodiments, during the step of vial pressurisation, some gas may also flow in the downstream direction A through the fluid check-valve 300. One or more components of the vented syringe 900 may be configurable to inhibit fluid flow in the downstream direction A during the vial pressurisation step. This may increase the proportion of gas which flows out of the fluid opening 923 during the vial pressurisation step. For example, in some embodiments, the fluid flow inhibition member 400 may be configurable, prior to performing the vial pressurisation step, to inhibit fluid flow in the downstream direction A. In other embodiments, the fluid flow inhibition member 400 may permit fluid flow during the vial pressurisation step.

In some embodiments, fluid flow through the fluid flow path 210 may be inhibited by means other than the fluid flow inhibition member 400. In some embodiments, the vented syringe 900 may comprise a secondary fluid flow inhibition member. The secondary fluid flow inhibition member may be configured, or configurable, to selectively inhibit flow within the fluid flow path 210. For example, where the fluid flow inhibition member 400 comprises a passive valve, a secondary fluid flow member may allow for inhibiting of fluid flow in the fluid flow path 210 under conditions where the fluid flow inhibition member 400 may permit fluid flow (such as in a vial pressurisation step, for example). The secondary fluid flow inhibition member may be configured, or configurable, to seal the fluid flow path. For example, the secondary fluid flow inhibition member may be configurable between an open configuration in which fluid flow is substantially uninhibited and a closed (or sealing) configuration in which fluid flow is inhibited. The secondary fluid flow inhibition member may be positioned, or positionable, downstream of the fluid flow inhibition member 400.

The secondary fluid flow member may comprise, for example, a releasable or breakable seal, a plug (such as a rubber stopper or other suitable plug), a flexible wall, a solid or flexible cover. The secondary fluid flow inhibition member may be actuable, for example electronically actuable. In other embodiments, the secondary fluid flow inhibition member may be configured to be selectively applied and/or actuated by a user.

In some embodiments, the piston 100 may be configured to allow selective sealing of the downstream end 212 of the fluid flow path 210 by a user. For example, in the illustrated embodiment, a user's thumb 40 may be placed across the downstream end 212 of the fluid flow path 210 to seal the fluid flow path 210 and thus inhibit fluid flow in the downstream direction A, during the vial pressurisation step. The user's thumb 40 in this instance may be considered to represent a secondary fluid flow inhibition member which is selectively applicable by a user.

In some applications, vial contamination may be an issue due to contaminants or lack of sterility in the ambient air. In some such applications, the piston 100 may be first displaced in the downstream direction A to cause gas 10 to flow from the downstream section 922 to the upstream section 921, through the fluid check-valve 300, so that the syringe is pre-aspirated with sterile air. That is, displacement of the piston 100 may draw gas 10 into the upstream section 921 of the vented syringe barrel 910 through the fluid check-valve 300. The fluid opening 923 may then be inserted into the vial, the valve 400 closed and the plunger 100 displaced slightly in the upstream direction B. This causes the syringe 900 to pressurise the vial with clean air, reducing the potential for contamination of the vial.

After pressurisation of the vial, the vented syringe 900 may be configured to allow fluid flow in the downstream direction A. For example, the fluid flow inhibition member 400 may be configured to allow fluid flow in the downstream direction A. Alternatively or additionally, a secondary fluid flow inhibition member (if present) may be removed or otherwise configured to allow fluid flow in the downstream direction A. In the illustrated embodiment, as shown in FIG. 6-3, the user's thumb is removed from the downstream end 212 of the fluid flow path 210, allowing fluid to flow in the downstream direction A. Under the increased pressure (relative to ambient) introduced to the vial in the step of FIG. 6-2, liquid 20 from the vial may flow into the upstream section 921 of the vented syringe 900.

As the liquid 20 flows into the upstream section 920 of the vented syringe, gas 10 flows along the fluid flow path 210 from the upstream section 921, through the fluid check valve 300, and is vented out of the downstream end 212 of the hollow plunger shaft 250 of the piston 100. In some cases, if sufficient pressure has been introduced to the vial, the liquid 20 may flow to the level of the fluid check valve 300, wetting the hydrophilic membrane 310. The hydrophobic membrane 320 inhibits the liquid from passing the fluid check-valve 300. Further flow of liquid 20 into the upstream section 921 of the vented syringe 900 may push the stopper 500, together with the piston 100, in the downstream direction A, as shown in FIG. 6-4, for example.

In some cases, for example if the fluid did not reach the level of the fluid check-valve 300 under the pressure from the vial, the piston 100 may be depressed in the upstream direction B to further remove bubbles of gas from the upstream section 921 of the vented syringe 900.

If required, further liquid 20 may be aspirated into the vented syringe 900 by displacing the piston 100 in the downstream direction A until a desired quantity of liquid 20 has entered the upstream section 921. The vented syringe 900 may then be moved to withdraw the needle cannula 800 from the vial. Liquid 20 may be dispensed from the vented syringe when required by depressing the piston 100 to force the liquid 20 from the end 801 of the needle cannula (to inject a liquid medicament into a patient's body, for example).

Vented syringes according to the present disclosure may reduce wastage when administering medications, as they allow the user to expel gas bubbles while sampling medication from the vial rather than after drug has already been removed.

Further, vented syringes according to embodiments of the present invention may be filled and operated without tipping or flipping the vented syringe. This may minimise the formation of bubbles and may prevent needle stick injuries that can occur when drawing fluid from an inverted vial.

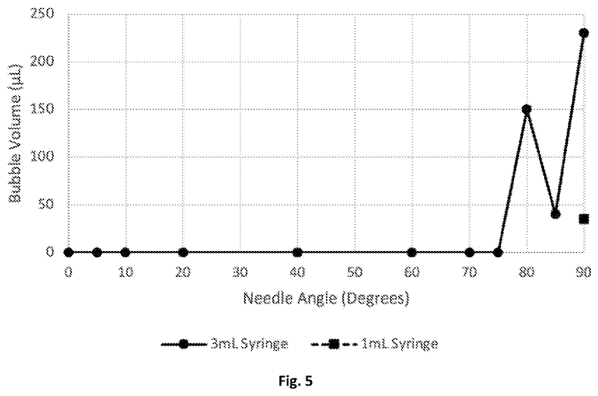

FIG. 5 demonstrates that air bubbles from the upstream section of the syringe barrel may be evacuated at a wide variety of angles of operation. This may allow the user to adopt a variety of positions when extracting medication and purging gas, increasing the ergonomic suitability of the vented syringe.

### Pre-Filled Syringes

Pre-filled syringes are an increasingly popular method of packaging medication. This may be due to several factors, including:
pre-filled syringes may be simple and convenient to use;
pre-filled syringes may limit overfill, reducing medication wastage;
pre-filled syringes may come pre-labelled reducing wastage and the potential of administrative errors.

Minimising wastage is especially important in the manufacturing of expensive medications such as biologics. Biologic drugs such as monoclonal antibodies are so expensive as to make the cost of packaging negligible. As such, even small decreases in overfill may offer appreciable cost savings.

Pre-filled syringes are typically manufactured in a multistage process by independent companies. Once the piston, piston seal and syringe barrel have been manufactured and sterilised they are packed in specialised tubs and bagged as appropriate for “fill and finish”.

“Fill and finish” is the process by which the syringe is filled, assembled, tested, individually labelled and packed for shipment to suppliers. FIG. 4A shows a simplified method of an automatic filling procedure for a conventional syringe. Generally, a syringe barrel, such as shown in FIG. 4A-1, is unpacked and loaded into an auto filling machine. An appropriate quantity of medicament liquid is then precision dosed into the syringe barrel, through an opening at either end of the syringe as shown in FIG. 4A-2. A stopper is then loaded into the syringe barrel, as shown in FIG. 4A-3, followed by a piston shaft, as shown in FIG. 4A-4. The complete syringe assembly is then tested and packaged. The pre-filled syringe may then be packed and stored. Medicament may be dispensed from the syringe, when required, by depressing the plunger rod to drive the stopper along the barrel, forcing medicament from the fluid opening at the distal end of the syringe, as shown in FIG. 4A-5.

Medications can be sensitive to contaminants generated during packaging and syringe manufacturing. This is especially true for biologics like monoclonal antibodies which are often prone to aggregation. Protein aggregation alters protein binding kinetics rendering the drug unsuitable for administration. As a result, a significant amount of testing is dedicated to ensuring drug packaging and storage will not result in aggregation for most biotherapeutics.

Syringe filling typically requires inert, sterile surfaces and accurate volumetric dosing. Biotherapeutics such as monoclonal antibodies are highly sensitive to mechanical stress and particulate contamination. This is because monoclonal antibodies are typically injected subcutaneously and at high dosage. This route of administration requires a high concentration in solution resulting in significant risk of aggregation. Therefore, it is important that the filling procedure for such medications avoids mechanical stresses and particulate contamination as each of these promote aggregation.

Diaphragm, peristaltic and rotating piston pumps are commonly used to pre-fill syringes. However, the use of these pumps risks plastic or metal particulate contamination due to mechanical degradation of process components. Otherwise these issues can be addressed using pressure/time filling machines which avoid these mechanical stresses and contamination risks but require precise physical characterisation of fluid viscosity to accurately dose each syringe.

Medications such as those intended for intravenous injection, or those sensitive to oxygen cannot be packaged with air bubbles and are stoppered using a vacuum method. However, such methods risk damaging the syringe barrel, creating particulate contaminants which may ruin the medication.

Vented syringes according to embodiments of the present disclosure may ameliorate one or more of the shortcomings associated with the conventional pre-filled syringe manufacturing processes. Vented syringes such as those described herein may allow for filling techniques that avoid significant sources of contamination and eliminate the complexity associated with syringe stoppering. Vented syringes according to embodiments of the present disclosure may allow for control of dosing volume using only the mechanical design of the vented syringe and the position of the piston.

A method of filling a vented syringe according to an embodiment of the present disclosure is shown in FIG. 4B. In FIG. 4B-1, a vented syringe 900 including a vented piston 100 pre-loaded into the vented syringe barrel 910 is unpacked and may be loaded into an auto filling machine. Liquid 20 is then dosed into the vented syringe through a fluid opening 923, as shown in FIG. 4B-2. As the liquid 20 flows through the vented syringe barrel 910, gas 10 within the upstream section 921 is evacuated through the vented piston 100 in a downstream direction. Once the liquid 20 reaches the fluid check-valve 300, the wetted fluid check-valve “locks”. If the piston 100 is held in a temporarily fixed position relative to the vented syringe barrel, dosing of further liquid 20 into the vented syringe 900 will be inhibited. As such, the dose of liquid 20 (medication) may be determined by the dimensions of the vented syringe barrel 910 and the position of the piston 100 within the vented syringe barrel 910. FIG. 4B-3 shows the completed pre-filled vented syringe 900, which may then be subjected to quality control testing and packed as required.

As such, vented syringes according to embodiments of the present disclosure may allow for an accurate fill volume to be obtained without the use of a pump and without an accurate model of fluid viscosity. In addition, vented syringe stoppering may be performed by the supplier, meaning that fill and finish operations may be achieved using potentially simpler (and therefore potentially more reliable) filling machines.

Once filled, a syringe must retain its contents until dispensing is required. Escape of fluid may lead to inaccurate dosing, wastage or exposure of a user to a dangerous substance. Vented syringes according to embodiments of the present disclosure prevent liquid loss from the vented syringe by the strong water-repelling pressure of the hydrophobic membrane 330. Hydrophilic membranes on their own, or alternatives such as paper filters, will slowly leak fluid due to capillary pressure. However, the liquid breakthrough resistance of a hydrophobic membrane is more than an order of magnitude larger than that of a hydrophilic membrane. The combination of a hydrophilic porous material 310 and hydrophobic porous material 320 employed in the presently disclosed vented syringe inhibits liquid escape through fluid check-valve. This may allow for simple loading of liquid into the vented syringe, while also preventing liquid loss and protecting the user against exposure to toxic fluids.

### Microfluidic Devices

Fluid handling at the small scale of microfluidic devices presents challenges. Microfluidic applications are especially sensitive to the presence of gas bubbles in solution, on surfaces or stuck in channels for a variety of reasons, including:
gas bubbles can prevent accurate dosing of reagents;
gas bubbles can significantly obstruct flow;
gas bubbles can damage sensitive compounds and obstruct surface interactions.

A microfluidic device may be used to measure rheological properties of blood. The present applicant's earlier filed patent applications PCT/GB2017/053393 and PCT/AU2020/050902 disclose examples of such devices. By controlling the flow rate of a sample fluid within a measurement section of the device, viscosity and shear rate can be calculated from a suitable fluidic device. For example, the fluidic device may comprise a pumping apparatus to control the flowrate of the sample flowing in the measurement unit where the pressure drop in a channel of defined dimensions is measured. With a known pressure drop, a known flow rate, and known channel dimensions, the viscosity and shear rate can be calculated. The flowrate is controlled via a syringe pump and the pressure drop across the measurement channel is performed using differential pressure sensors. To measure the full spectrum of viscosity properties of non-Newtonian fluids such as blood (i.e. that vary with the shear rate), the flowrate may be varied progressively over time following a sinusoidal pattern.

However, if a gas bubble gets trapped in the fluidic system between the syringe and the measurement channel, the gas bubble would get compressed and de-compressed due to the change in flowrate applied and subsequent variable pressure experienced throughout the fluid line. The bubble would therefore have its volume decreased and increased due to the pressure change. In turn, this would introduce a significant difference between the flowrate imposed by the movement of the syringe pump and the actual flowrate of the sample passing in the fluid line where the pressure difference is measured. Since the flowrate considered in the calculation of the viscosity is defined solely by the movement of the piston of the syringe pump, the aforementioned difference in flowrate between volumetric change in the syringe and the flowrate of the liquid in the channel where the pressure is measured generates an unacceptable error in the calculated viscosity. This error would therefore invalidate the measurement. Additionally, if a bubble is trapped within the measurement portions of a channel, less liquid than expected would be measured and/or the effective cross-section of the channel would be reduced. This creates significant errors in the calculated viscosity and/or invalidate calculations for the viscosity that use the physical/absolute cross-sectional size of the channel. This highlights the importance of removing bubbles from the system.

The gas venting functionality of vented syringes according to embodiments of the present disclosure may allow a user to remove bubbles from fluid samples before they are injected into the microfluidic device. This may be relevant for samples which are dangerous, mechanically sensitive, prone to foaming or are highly viscous.

Additionally, some protocols may require sample collection from a microfluidic device. Vented syringes according to embodiments of the present disclosure may allow a user to insert the vented syringe into a receiving port with the piston positioned at a desired volume and remove the vented syringe once the appropriate volume has been generated.

Such functionality may be applicable in a number of scenarios, for example, the administration of sensitive and expensive customised therapeutics such as microfluidically generated gene therapies. In some scenarios, it is important to eliminate gases from samples, such as when analysing liquid solutions containing volatile compounds. As such, vented syringes according to embodiments of the present disclosure may allow for simple protocols when working in sensitive applications.

Vented syringes according to the present disclosure may also have potential application in the integration of vented closures and pumps into microfluidic chips. Vents integrated into microfluidic chips may allow for changes in pressure, volume and composition within the chips. For example, a vented closure allows reactions that produce gases to occur ‘on chip’ without endangering downstream processes. In addition, a vented syringe piston allows for volumetric pumping action to occur within a piston cylinder embedded within the chip material.

### Fluid Dispensing

Vented syringes according to embodiments of the present disclosure may be useful in fluid dispensing applications. Automatic dispensing of fluid volumes in the sub 10 μL range is complicated by capillary and adhesion forces which limit accurate volumetric dosing and enhance problems that can be created by gas bubbles. As a result, methods of automated fluid handling such as air displacement pipettes become increasingly inaccurate the smaller or more viscous the volume. Low dead space syringes and/or positive displacement pipettes may be preferred for bubble free, viscous and small volume fluid handling applications. However, for high throughput or time-dependent dosing these devices may not be suitable, as low dead space syringes still require priming and positive displacement pipettes are restricted to small total volumes. Therefore, in applications such as high-performance liquid chromatography, where fluid lines require priming, vented syringes such as those disclosed herein may provide for priming of fluid lines more easily and simply, via vacuum.

Vacuum priming may be performed by applying a vacuum to pull liquid into a fluid line starting at the outlet. Thus, the entire fluid line can be purged of gas from beginning to end in a single step. This saves time, effort and materials on the part of the user and has the potential to be a more reliable method than pushing liquid through the system in the forward direction.

Vented pistons according to the present disclosure may also be useful in automated fluid (or fluids) handling. Vented pistons according to embodiments of the present disclosure may be incorporated into syringe pumps to create an automated fluid handling device, or robot. The automated fluid handling device may include one or more control systems for controlling movement of the piston within the fluid conduit and/or controlling the configuration of the fluid flow inhibition member to allow or inhibit fluid flow within the fluid flow path. The automated fluid handling device may also control positioning of the fluid conduit (e.g., syringe) in relation to a fluid source (such as a vial). The automated fluid handling device may automatically run a protocol to load fluid, evacuate air, obtain the desired fluid volume, move to an outlet and dispense a controlled volume of the fluid(s).

Vented pistons according to the present disclosure may be relatively simple to manufacture and/or operate. This may be due to a small number of individual components and/or due to the axially fixed arrangement of one or more components, such as the vented plunger shaft, stopper, fluid check-valve and/or fluid flow inhibition member, relative to one another.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A vented piston receivable within a fluid conduit, the piston comprising:
a plunger body, the plunger body defining a fluid flow path extending between an upstream end and a downstream end;
a fluid check-valve disposed across the fluid flow path, the fluid check-valve configurable to inhibit passage of gas from the downstream end to the upstream end of the fluid flow path and to inhibit passage of liquid from the upstream end to the downstream end of the fluid flow path; and
a fluid flow inhibition member disposed across the fluid flow path to inhibit fluid flow within the fluid flow path,
wherein the fluid check-valve is axially fixed relative to the plunger body.

2. The vented piston according to claim 1, wherein the fluid flow inhibition member is configurable to selectively inhibit fluid flow within the fluid flow path.

3. The vented piston according to claim 2, wherein the fluid flow inhibition member is configurable to permit fluid flow from the upstream end to the downstream end of the fluid flow path and to inhibit fluid flow from the downstream end to the upstream end of the fluid flow path.

4. The vented piston according to any one of the preceding claims, wherein the fluid flow inhibition member is positioned downstream of the fluid check-valve.

5. The vented piston according to any one of the preceding claims, wherein the fluid flow inhibition member comprises a valve.

6. The vented piston according to claim 5, wherein the fluid flow inhibition member comprises a passive valve.

7. The vented piston according to claim 5, wherein the fluid flow inhibition member comprises an actuable valve.

8. The vented piston according to any one of the preceding claims, wherein the fluid conduit comprises an inner surface and wherein the piston forms a fluidic seal with the inner surface of the fluid conduit.

9. The vented piston of claim 8, wherein the plunger body is adapted to form a fluidic seal with the inner surface of the fluid conduit.

10. The vented piston of claim 8 wherein the piston further comprises a sealing member for forming a fluidic seal with the inner surface of the fluid conduit.

11. The vented piston of claim 10, wherein the sealing member comprises a stopper at an upstream end of the piston, wherein the stopper includes an axial channel in fluid communication with the fluid flow path.

12. The vented piston according to any one of the preceding claims, wherein the fluid check valve comprises:
a hydrophilic porous material; and
a hydrophobic porous material disposed adjacent the hydrophilic porous material
wherein one face of the hydrophilic porous material is in fluid communication with the upstream end of the fluid flow path, and one face of the hydrophobic porous material is in fluid communication with the downstream end of the fluid flow path, and
wherein the hydrophilic porous material is configured to retain liquid from the upstream end to inhibit passage of gas from the downstream end to the upstream end, and the hydrophobic porous material is configured to inhibit passage of liquid from the upstream end to the downstream end.

13. The vented piston according to claim 12, wherein the hydrophilic porous material is disposed upstream of the hydrophobic porous material.

14. The vented piston according to claim 12 or claim 13, wherein the plunger body comprises a retention body for retaining the hydrophilic material and the hydrophobic porous material within the plunger body, wherein the fluid flow path extends through the retention body.

15. The vented piston according to claim 14, wherein the retention body comprises a first part and a second part, connectable to each other to cooperatively retain the hydrophilic porous material and the hydrophobic porous material.

16. The vented piston according to claim 15, wherein the first part comprises a recess configured to receive the hydrophilic porous material and the hydrophobic porous material, and wherein the second part includes a projection receivable in the recess and configured to retain the hydrophilic porous material and the hydrophobic porous material in the recess.

17. The vented piston according to any one of claims 14 to 16, wherein the plunger body further comprises a plunger rod, wherein the retention body includes a coupling member for coupling the retention body to the plunger rod.

18. The vented piston according to claim 17, wherein the fluid flow path extends through the plunger rod.

19. The vented piston according to claim 18, wherein the coupling member fluidly connects the plunger rod to the retention body.

20. The vented piston according to any one of claims 14 to 19, wherein the retention body is integral with the plunger rod.

21. A vented syringe including the vented piston of any one of the preceding claims.

22. The vented syringe of claim 21, wherein the vented syringe includes a syringe barrel defining the fluid conduit and adapted to at least partially receive the piston,
wherein, when at least partially received in the syringe barrel, the piston divides the syringe barrel into an upstream section and a downstream section,
wherein the syringe barrel defines a fluid opening adjacent the upstream section.

23. A method of filling the vented syringe of claim 22, the method comprising:
immersing the fluid opening in a liquid;
displacing the piston in a downstream direction to draw liquid into the upstream section of the syringe barrel;
displacing the piston in an upstream direction, to cause gas to flow through the fluid flow path in the downstream direction.

24. The method of claim 23, wherein the piston is further displaced in the upstream direction to bring the fluid check-valve into contact with liquid.

25. The method of claim 23 or claim 24, further comprising, prior to displacing the piston in the downstream direction to draw liquid into the upstream section of the syringe barrel, displacing the piston in an upstream direction to force gas in the upstream section to flow out the fluid opening.

26. The method of any one of claims 23 to 25, further comprising, prior to immersing the fluid opening in the liquid source, displacing the piston in the downstream direction to cause gas to flow from the downstream section into the upstream section.

27. The method of any one of claims 23 to 26, further comprising:
displacing the piston in a downstream direction to draw further liquid into the upstream section of the syringe barrel.

28. The method of any one of claims 23 to 27, further comprising, prior to displacing the piston in a downstream direction to draw liquid into the upstream section of the syringe barrel, configuring the fluid flow inhibition member to inhibit fluid flow from the downstream end to the upstream end of the fluid flow path.

29. The method of any one of claims 23 to 28, further comprising, prior to displacing the piston in an upstream direction to cause gas to flow through the fluid flow path in the downstream direction, configuring the fluid flow inhibition member to allow fluid flow from the upstream end to the downstream end of the fluid flow path.

30. The method of claim 29, wherein configuring the fluid flow inhibition member to inhibit fluid flow comprises configuring the fluid flow inhibition member to form a fluidic seal across the fluid flow path.

31. A method of filling the vented syringe of claim 22, the method including:
connecting the fluid opening with a liquid source;
causing the liquid to flow into the upstream section of the syringe barrel such that gas within the upstream section of the syringe barrel flows in a downstream direction through the fluid flow path.

32. The method of claim 31, wherein a position of the piston within the syringe barrel is selected to define a predetermined fill volume.

33. A fluidic system comprising:
a fluid conduit comprising an inner surface; and
a vented piston according to any one of claims 1 to 20, wherein the piston is at least partially received within the fluid conduit and forms a fluidic seal with the inner surface of the fluid conduit to separate the fluid conduit into an upstream section to contain a gas and a liquid, and a downstream section to receive the gas.

34. The fluidic system according to claim 33, further comprising an automated motion control system configured to engage the piston to control the movement of the piston.

35. The fluidic system according to claim 34, wherein the automated motion control system is configured to:
actuate the piston to move the piston in a downstream direction to draw a fluid into the fluid conduit;
actuate the piston to move the piston in an upstream direction to evacuate gas from the upstream section of the fluid conduit;
actuate the piston to move the piston in the downstream direction to draw further fluid into the upstream section to obtain a desired fluid volume; and
actuate the piston to move the piston in the upstream direction to dispense a controlled volume of the fluid.

36. The fluidic system of claim 33 or claim 34, further comprising an automated filling system, wherein a position of the piston within the fluid conduit is selected to define a predetermined fill volume.

37. The vented piston of any one of claims 1 to 20, or the vented syringe of claim 21 or claim 22, wherein the fluid flow inhibition member is axially fixed relative to the plunger body.

38. The steps, features, integers, compositions and/or compounds disclosed herein or indicated in the specification of this application individually or collectively, and any and all combinations of two or more of said steps or features.
